(19) United States
(12) Patent Application Publication
(10) Pub. No.: US 2022/0280051 A1
(43) Pub. Date: Sep. 8, 2022

(54) IN-EAR WEARABLE DEVICE

(71) Applicants: Arizona Board of Regents on behalf of Arizona State University, Scottsdale, AZ (US); The United States of America as represented by the Secretary of the Navy, Indian Head, MD (US)

(72) Inventors: Sangram Redkar, Mesa, AZ (US); Kevin Nichols, Gilbert, AZ (US); Prabha Dwivedi, Port Tobacco, MD (US)

(21) Appl. No.: 17/685,914

(22) Filed: Mar. 3, 2022

Related U.S. Application Data
(60) Provisional application No. 63/156,997, filed on Mar. 5, 2021.

(51) Int. Cl. A61B 5/0205; A61B 5/00; G16H 40/67

(52) U.S. Cl. CPC A61B 5/02055; A61B 5/0022; G16H 40/67

(57) ABSTRACT

Systems and methods for monitoring physiological parameter(s) and environmental condition(s) of a subject include an in-ear wearable apparatus. The in-ear wearable apparatus includes a housing, a controller coupled to the housing, a first plurality of physiological sensors coupled to the housing and configured to detect a plurality of physiological parameters, and at least one environmental sensor coupled to the housing and configured to detect at least one environmental condition.

### CROSS REFERENCE TO RELATED APPLICATIONS

This application claims priority to, and the benefit of, U.S. Provisional Patent Application Ser. No. 63/156,997, entitled “IN-EAR WEARABLE DEVICE,” filed on Mar. 5, 2021. The content of the foregoing application is hereby incorporated by reference (except for any subject matter disclaimers or disavowals, and except to the extent of any conflict with the disclosure of the present application, in which case the disclosure of the present application shall control).

### FEDERALLY FUNDED RESEARCH OR DEVELOPMENT

This invention was made with government support under grant number CB10787 awarded by the Defense Threat Reduction Agency (DTRA) and grant number N00174-20-1-0002 awarded by Naval Sea Systems Command (NAVSEA). The government has certain rights in the invention.

### TECHNICAL FIELD

The present disclosure relates to wearable devices, and in particular to in-ear wearable devices.

### BACKGROUND

Military personnel commonly train and/or operate under extreme conditions and in dangerous environments. During battle there are numerous causes of mortality, both direct and indirect, which, if avoided, would spare many lives. These causes include, but are not limited to: (1) fratricide (deaths from friendly fire); (2) deaths resulting from extreme environmental conditions; (3) deaths of medics and others during attempts to rescue those who are already dead or who are mortally wounded; (4) delay in locating casualties beyond the short period during which treatment most likely will be effective; (5) inadequate data to guide optimum initial evaluation by medical personnel in the field; (6) difficulty interpreting the available data in the stress of battle; (7) difficulty in maintaining consistent reevaluation during transport to and through higher levels of care; and (8) difficulty during peacetime in acquiring and maintaining combat trauma treatment skills by medical personnel. It is believed that if some or all of these problems were adequately addressed, a considerable number of lives could be saved during combat situations.

### SUMMARY

In an exemplary embodiment, an in-ear wearable apparatus for monitoring physiological and environmental parameters of a person, comprises a housing, a controller coupled to the housing, a first plurality of physiological sensors coupled to the housing and configured to detect a plurality of physiological parameters, and at least one environmental sensor coupled to the housing and configured to detect at least one environmental condition.

In various embodiments, the controller is configured to transmit the plurality of physiological parameters and the at least one environmental condition to a centralized computer.

In various embodiments, the first plurality of physiological sensors comprises at least two of the following: an IMU sensor, a pulse oximetry sensor, a GSR sensor, an EMG Sensor, an EKG sensor, and an EEG sensor.

In various embodiments, the at least one environmental sensor comprises at least one of the following: a barometer/humidity sensor, a gas sensor, and a radiation sensor.

In various embodiments, the first plurality of physiological sensors comprises the IMU sensor, the pulse oximetry sensor, the GSR sensor, the EMG Sensor, the EKG sensor, and the EEG sensor.

In various embodiments, the at least one environmental sensor comprises the barometer/humidity sensor, the gas sensor, and the radiation sensor.

In various embodiments, the in-ear wearable apparatus further comprises an ear-mold and an in-ear sensor mounted to the ear-mold.

In various embodiments, the in-ear sensor comprises at least one of a pulse oximetry sensor, a temperature sensor, and a heart rate sensor.

In various embodiments, the housing is wearable behind an ear of the person ear and the ear-mold is wearable in the ear of the person.

In various embodiments, the in-ear wearable apparatus further comprises a battery coupled to the housing, the battery configured to power the controller.

In various embodiments, the in-ear wearable apparatus further comprises a flexible printed circuit board comprising an integrated pulse oximeter sensor.

In various embodiments, the flexible printed circuit board is mounted to an ear-mold of the in-ear wearable apparatus.

In various embodiments, the flexible printed circuit board further comprises an integrated electrocardiogram sensor connector.

In various embodiments, the flexible printed circuit board further comprises an integrated motion sensor.

A method of monitoring a physiological parameter and an environmental condition of a subject via an in-ear wearable apparatus, wherein the in-ear wearable apparatus includes a housing, a processor attached to the housing, a plurality of physiological sensors, and at least one environmental sensor, is disclosed. The method comprises obtaining physiological information from the subject via the plurality of physiological sensors, wherein the physiological information comprises at least one of the following: 3 axis head acceleration information, 3 axis rotation rate information, head orientation (roll, pitch, yaw) information, head angular acceleration information, cerebral oxygen saturation information, heart rate information, heart rate variability information, breathing rate information, breathing rate variability information, body temperature information, and vibration information, and obtaining environmental information from the subject via the at least one environmental sensor, wherein the environmental information comprises at least one of the following: pressure information, attitude information, humidity information, temperature information, radiation information, and toxic gas information.

In various embodiments, the method further comprises transmitting the physiological information and the environmental information to a device remotely located from the subject.

In various embodiments, the method further comprises processing the physiological information and the environmental information to determine at least one of the following: a position of the subject, a cognitive distress of the subject, a physical distress of the subject, an emotional distress of the subject, a cardiac distress of the subject, a pulmonary distress of the subject, a muscular distress of the subject, and an environmental toxicity.

The foregoing features, elements, steps, or methods may be combined in various combinations without exclusivity, unless expressly indicated herein otherwise. These features, elements, steps, or methods as well as the operation of the disclosed embodiments will become more apparent in light of the following description and accompanying drawings.

### BRIEF DESCRIPTION OF THE DRAWINGS

With reference to the following description and accompanying drawings:

FIG. 1A illustrates an assembly view of an EWD, in accordance with various embodiments.

FIG. 1B illustrates a rear view of the EWD of FIG. 1A, in accordance with various embodiments.

FIG. 1C illustrates the EWD worn by a user in testing, in accordance with various embodiments.

FIG. 1D illustrates the EWD worn by a user with snap electrodes worn by the user electrically coupled to the EWD, in accordance with various embodiments.

FIG. 2A illustrates a perspective view of an EWD with GSR electrodes worn by a user, in accordance with various embodiments.

FIG. 2B and FIG. 2C illustrate a prototype GSR and the resulting sensor data, respectively, in accordance with various embodiments.

FIG. 3A and FIG. 3B illustrate a prototype EMG sensor and preliminary testing results, respectively, in accordance with various embodiments.

FIG. 4A illustrates hardware/electronics for an exemplary prototype ECG sensor, in accordance with various embodiments.

FIG. 4B illustrates electrodes (leads) attached behind the ear, in accordance with various embodiments.

FIG. 4C illustrates the waveform (sinus rhythm) obtained from the sensor of FIG. 4A and

FIG. 4B, in accordance with various embodiments.

FIG. 5A illustrates a single lead EEG system, in accordance with various embodiments.

FIG. 5B illustrates the EEG signal and power spectral analysis of the EEG signal of the single lead EEG system of FIG. 5A, in accordance with various embodiments.

FIG. 6A illustrates a prototype MEMS Barometer/Humidity sensor, in accordance with various embodiments.

FIG. 6B illustrates a prototype MEMS air quality sensor, in accordance with various embodiments.

FIG. 7 illustrates a prototype and testing of a radiation detection sensor, in accordance with various embodiments.

FIG. 8 illustrates a system for monitoring personnel physiological parameters and environmental conditions using one or more EWDs, in accordance with various embodiments.

FIGS. 9A, 9B, and 9C illustrate a prototype earpiece for an EWD system, in accordance with various embodiments.

FIGS. 10A, and 10B illustrate a front-side view and a back-side view, respectively, of a flexible printed circuit board (PCB) with integrated pulse oximeter sensor and EKG sensor, in accordance with various embodiments.

FIG. 11 illustrates a screenshot of electrocardiogram sensor electrodes connected to an EWD earpiece, in accordance with various embodiments.

### DETAILED DESCRIPTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from principles of the present disclosure.

For the sake of brevity, the connecting lines shown in various figures contained herein are intended to represent exemplary functional relationships and/or physical couplings between various elements. It should be noted that many alternative or additional functional relationships or physical connections may be present in in-ear wearable devices.

The present disclosure relates to systems and methods for remote monitoring of personnel, and especially to a system for monitoring the well-being of military personnel on the battlefield and during training exercises. Such monitors could be extremely valuable for military personnel who commonly train under extreme weather conditions, and in other dangerous environments. As will be apparent from the accompanying specification, the military version of the device can easily be modified for use in civilian applications, such as medical care and medical monitoring of personnel working under adverse environmental conditions, such as firefighters, law enforcement personnel, seamen, field maintenance personnel, athletes, etc. For example, during peacetime, monitoring chemical-biological (CB) status could be beneficial for people exposed to hazardous occupational and/or environmental conditions, such as law enforcement, firefighters, sailors, mountaineers, athletes and the like.

Commercial-off-the-shelf wearable sensor technologies fall short of various mission needs.

Thus, there exists an unmet mission requirement to create novel and high quality products that will help to 1) detect, identify, and mitigate chemical, biological, radiological, nuclear, and explosives (CBRNE) weapons, and 2) assess warfighter health and readiness through autonomic and biochemical signature measurements. Wearable technology of the present disclosure has the potential to make each subject (e.g., soldier) a chemical, biological, radiological, nuclear, and high-yield explosives point sensor, giving commanders an unprecedented ability to conduct continuous surveillance in real time over wide areas of operation with little to no interference by equipment. By monitoring physiological variables, those overseeing exercises can monitor the soldier, etc., and withdraw him/her from the exercise if it appears that harm is likely. By providing accurate information about CB status of each individual, as well as communications equipment to convey the information to remote locations, a system for monitoring personnel could save many lives.

An in-ear wearable device (EWD) of the present disclosure detects negative physiological effects such as distress that can be expressed as symptoms of a combat soldier's cerebral health and physical status. In embodiments, an EWD of the present disclosure has embedded sensors to detect potentially hazardous external CB stimuli. In embodiments, an EWD of the present disclosure functions primarily by the housed hardware integrating non-invasive sensors for tracking electro-activities at the dermal layer occurring internally within the user's body. In embodiments, contactless sensors detect gas concentrations (e.g., in parts per million (ppm)) in the surrounding environment using the respective sensing elements. In embodiments, most or all sensors are based on micro-electro-mechanical systems (MEMS). In embodiments, the data signals are collected electronically then transmitted wirelessly for analysis in real-time utilizing a software algorithm on a mobile device platform, e.g. a smartphone. An EWD of the present disclosure may comprise a commercial-ready, wearable tech of small dimensions, lightweight with multiple sensors, and specific/accurate measurements for real-time readouts. Sensor data of an EWD of the present disclosure may be more accurate and reliable due to the ability to monitor a soldier's cerebral/physical state, providing alerts of readiness prior to engagements during combat or routine orders, but overall tracking their wellbeing and assessing their performance capabilities which can be further indicators during monitoring.

Features of interest of the disclosed EWD include, but are not limited to: (1) A novel, EWD for a combat solider; (2) Detection of negative distress using embedded sensors and potentially hazardous chemical-biological (CB) agents to a subjects physical status; (3) Functionality/novelty: integrated non-invasive sensors for tracking electro-activities at the dermal layer of the user's body; (4) Multiple sensors for tracking environmental gas concentrations (ppm) based on MEMS; (5) Challenges/barriers: delivering a commercial-ready, wearable tech of smaller dimensions, lighter weight for real-time app readouts; (6) Advantages/novelty: the ability to monitor a soldier's cerebral/physical state, alertness level, assessing their performance capabilities, etc.; and (7) Potential applications: in the military defense sectors for soldiers of various positions along with the private sectors, e.g. workers and athletes.

The EWD of the present disclosure combines hardware and software for continuous monitoring of physiological signals. The details about the hardware and software are provided herein.

### Hardware

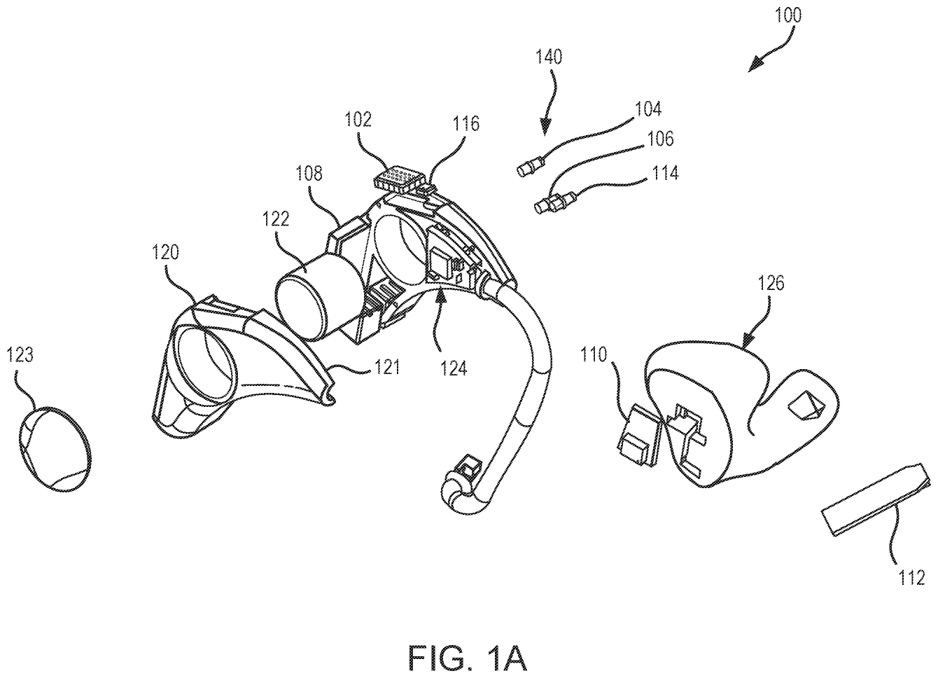

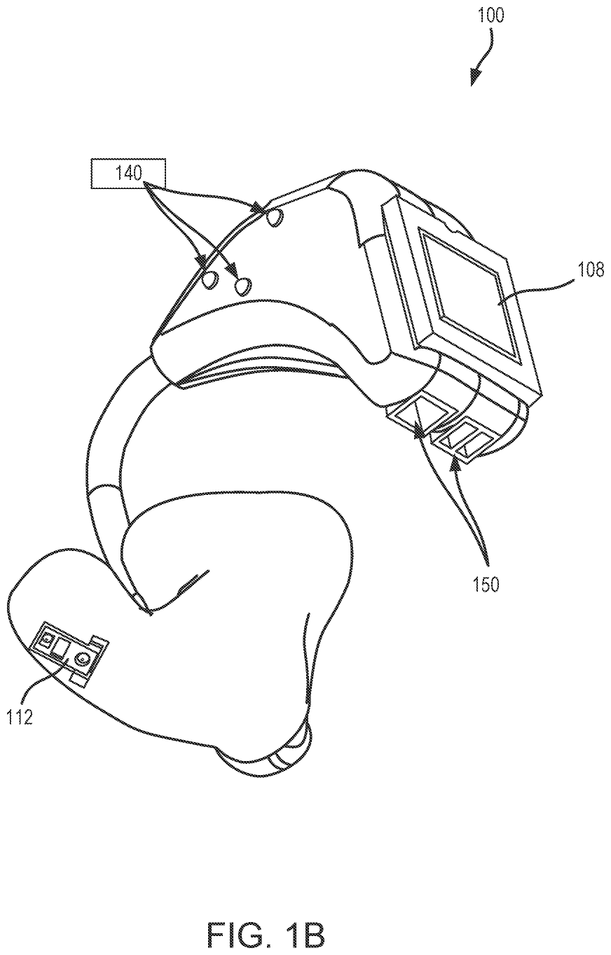

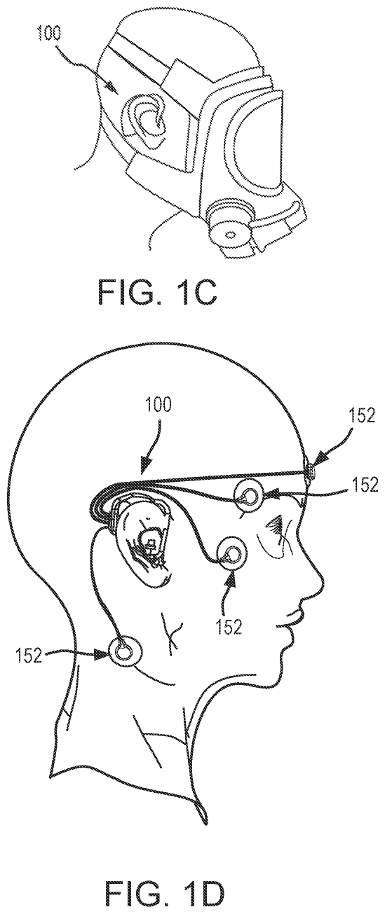

In embodiments, EWD hardware integrates many sensors in a small package, as shown in FIG. 1A through FIG. 1D. With reference to FIG. 1A, an assembly view of an EWD 100 of the present disclosure is illustrated, in accordance with various embodiments. EWD 100 may include various physiological and/or environmental sensors including a volatile organic compounds (VOC) sensor 102, an electromyography (EMG) sensor 104, a galvanic skin response (GSR) sensor 106, a radiation sensor 108, an inertial measurement unit (IMU) sensor 110, an in-ear sensor 112, a heart rate sensor 114 (e.g., an electrocardiogram (EKG or ECG) sensor). In-ear sensor 112 may include a pulse oximetry sensor (e.g., a pulse oximeter) and/or a temperature sensor. EWD 100 may further include a pressure sensor 116 (e.g., a barometer). Various sensors integrated into the EWD hardware may be commercially available sensors. It should be appreciated that the “integration” of various sensors that are used for measuring internal “physiological” and external “environmental” signals is a novel aspect of the present disclosure.

With combined reference to FIG. 1A and FIG. 1B, EWD 100 may include a plurality of electrodes 140. Each electrode 140 may comprise a sensor, for example a first electrode comprising the EMG sensor 104, a second electrode comprising the GSR sensor 106, and/or a third electrode comprising the EKG/ECG sensor 114. Although illustrated as comprising three electrodes, it should be understood that more or less electrodes may be provided depending on the desired number of electrode sensors. For example, a fourth electrode may be provided comprising an electroencephalography (EEG) sensor. Each sensor may comprise one or more electrodes. Each electrode may comprises one or more sensors (i.e., EWD 100 may use a single electrode (or electrode pair) for sensing one or more parameters. The plurality of electrodes 140 may be positioned such that the electrodes 140 contact a user's skin when the EWD 100 is worn by the user. Any of the electrode sensors of the present disclosure may be configured as one or more electrodes 140.

The inventors of the present disclosure have designed custom printed circuit boards (PCBs) and firmware that can acquire data from various sensors reliably and consistently. In embodiments, the EWD can wirelessly transmit data to a mobile device or host computer. EWD 100 may comprise electronics 124 which includes one or more controllers (e.g., processors) and one or more tangible, non-transitory memories capable of implementing digital or programmatic logic. Electronics 124 may further include an EEG sensor. In various embodiments, for example, the one or more controllers are one or more of a general purpose processor, digital signal processor (DSP), application specific integrated circuit (ASIC), field programmable gate array (FPGA), or other programmable logic device, discrete gate, transistor logic, or discrete hardware components, or any various combinations thereof or the like. Electronics 124 may control at least various parts of, and operation of various components of the EWD 100. Electronics 124 may include a transmitter for sending sensor data to a mobile device platform, (e.g. a smartphone located on the subject) which conveys sensor data to a device (e.g., a centralized computer for overseeing activity of the EWD user(s)) remotely located from the subject. Sensor data may be transmitted from the EWD 100 to the centralized computer in real time. EWD 100 may send raw sensor data to be analyzed by a separate computing device or may analyze raw data (e.g., in-sensor processing) and send processed sensor signals to the separate computing device. EWD 100 may further comprise a battery 122 for powering the various electronic components of EWD 100.

EWD 100 further comprises a housing 120. Electronics 124 may be disposed within housing 120. Housing 120 may comprise one or more seals 121 for sealing housing 120 to prevent contaminants, such as dust, water, sweat, etc. from entering housing 120. Housing 120 may include a battery cover 123 removably coupled to housing 120 for replacing the battery 122. In embodiments, housing 120 is mounted to the backside of a user's outer ear. EWD 100 may further comprise an ear-mold 126 (also referred to herein as an earpiece) for mounting various sensors in or partially within a user's ear canal. In embodiments, in-ear sensor 112 (comprising a pulse oximetry sensor, a temperature sensor, and/or an EKG sensor) is mounted to ear-mold 126. In embodiments, IMU sensor 110 is mounted to ear-mold 126.

FIG. 1C and FIG. 1D depict EWD 100 in use and installed on a user's ear. Preliminary specifications of EWD 100 are shown in Table 1. With momentary reference to FIG. 1B, EWD 100 may further include a plurality of connectors 150 for connecting at least one snap electrode to a user. With combined reference to FIG. 1B and FIG. 1D, at least one electrode 152 may be attached to a user (e.g., via an adhesive patch) wherein the electrode 152 is coupled (e.g., via at least one wire) to EWD 100 with the connector(s) 150. In this manner, the electrodes 152 are able to be placed onto the user spaced apart from EWD 100 for monitoring various physiological parameters of the user. Any of the electrode sensors of the present disclosure may be configured as one or more electrodes 152.

TABLE 1
Preliminary Specification of EWD

| Metrics | Threshold | | Objective | |
|---|---|---|---|---|
| Total weight (ear plug + | 40 | gm | 25 | gm |
| electronics) | | | | |
| Size (electronics box) | 25 mm diameter 10 mm | | 20 mm diameter 8 mm | |
| | thickness | | thickness | |
| Battery Life | 8 | hours | 8 | hours |
| Update Rate | 1 | Hz | 1 | Hz |
| Memory | 8 | GB | 8 | GB |

| | | |
|---|---|---|
| Interface for data download | Micro USB, Wireless | Micro-USB, Wireless |
| Accuracy (in High G | 95% | 99% |
| environment) | | |
| External Triggers | One (audio or hepatic | Two (audio and hepatic |
| capability | feedback) | feedback) |
| Cost (in quantities of 1000 | <$1000 per pair | <$500 per pair |
| units) | | |

| | |
|---|---|
| Physiological parameters | 3 axis head accelerations, 3 axis rotation rates, head |
| to be monitored | orientations (roll, pitch, yaw), head angular accelerations, |
| | cerebral oxygen saturations, heart rate, heart rate |
| | variability, breathing rate, breathing rate variability, body |
| | temperature, vibration. |
| Adverse events to be | GLOC, ALOC, Time for Useful Consciousness (TUC), |
| detected via physiological | hypoxia, hyperventilation, head ergonomics, thermal |
| signals | stress, excessive vibration, neck loading, fatigue, |
| | excessive/restricted neck motion, acceleration |
| | atelectasis, pulmonary distress, anxiety (based on |
| | berating rate and heart rate variation), bad posture |
| | leading to neck or shoulder pain, neck injuries. |

In embodiments, the EWD 100 contains physiological sensors shown in Table 2 and environmental sensors shown in Table 3.

TABLE 2
Physiological Sensors in EWD

| EWD | | | |
|---|---|---|---|
| To detect onset | | | |
| of multiple | | | |
| incapacitating | | | |
| physiological | | | |
| events due to | | | |
| CBRN | | Physiological | |
| exposure | Sensor | Parameter Measured | Symptoms |
| ✓ | IMU (Accelerometer, | Acceleration, angular | Fatigue, excessive |
| | Gyroscope, | velocity, elevation data | head/neck |
| | Magnetometer) | like total gain/loss | loading, spatial |
| | | ascent/descent, and | disorientation, g |
| | | elevation rate | loading |
| ✓ | Pulse Oximeter | Cerebral blood oxygen | Cardiac distress, |
| | | saturation, Heart rate | Pulmonary |
| | | and intervals, | distress, Hypoxia, |
| | | Breathing rate and | Hypo/hyper- |
| | | intervals, Body | ventilation |
| | | temperature | |
| ✓ | GSR Sensor (Galvanic | Skin resistance; skin | Muscular and |
| | Skin Response) | Conductance, Changes | Emotional |
| | | in electrical (ionic) | distress, Excessive |
| | | activity resulting from | sweating |
| | | changes in sweat gland | |
| | | activity | |
| ✓ | EMG Sensor | Muscle movement; | Muscular |
| | (Electromyography) | Burst of electrical | Distress/twitching, |
| | | activity which | Muscle spasm, |
| | | propagates through | and Eye irritation |
| | | adjacent tissue and | |
| | | bone and can be | |
| | | recorded from | |
| | | neighboring skin areas | |
| ✓ | EKG | Timing and shape of | Myocardial |
| | (Electrocardiogram) | the characteristic P-, | Infarction and |
| | | Q-, S-, and T- waves | Atrial Fibrillation |
| | | within the cardiac | |
| | | cycle | |
| ✓ | EEG | Event Related Potential | Attention, blink |
| | (Electroencephalogram) | (ERP), Alpha and theta | detection, mental |
| | | waveform, Peak alpha | effort and |
| | | frequency | alertness |

TABLE 3
Environmental Sensors in EWD

| | | | Sudden variations |
|---|---|---|---|
| | Barometer/Humidity | Pressure, Attitude, | in cabin |
| ✓ | Sensor | Humidity, Temperature | pressure/humidity |
| ✓ | Gas Sensor (e.g., | CO (~1 to 1000 ppm), | Environmental |
| | VOC sensor) | Ammonia (~1 to 500 ppm), | Toxicity |
| | | Ethanol (~10 to 500 ppm), | |
| | | H2 (~1 to 1000 ppm), and | |
| | | Methane/Propane/Iso- | |
| | | Butane (~1000++ ppm) | |
| ✓ | Radiation Sensor | α, β, γ, and X-ray radiation | Environmental |
| | | | Toxicity |

### Software

The data provided by sensor (raw data) can be contaminated by noise, sensor errors such as hysteresis, drift, thermal bias, etc., to mention a few. The inventors of the present disclosure have developed a software framework and algorithms that accept this raw data, perform signal processing, and provide meaningful results under varying external conditions and sensor characteristics.

The typical signal processing algorithm involves:

1. Removal of DC bias from the sensor;

### 2. Thermal Compensation;

3. Scale factor/cross axis determination;
4. Low pass filtering;
5. Compensation for undesired artifact via adaptive signal processing;
6. Fuzzy logic filtering for anomaly detection;
7. Signal separation algorithm to separate desired and undesired signals;
8. Signal integrity validation algorithms; and
9. Final signal output/result.

The result obtained in step 9 is used to detect physiological distress event as indicated by a single sensor. The output of all the sensors is fed to a central algorithm that “combines” individual sensor output selectively and provides feedback of type of distress (a) cardiopulmonary, (b) cognitive, (c) hypoxia, (d) stress and its relative intensity.

### Description and Operation

The final EWD system includes multiple sensors. The following sections discuss different types of sensors in EWD and their application to real-time physiological monitoring.

1. Pulse Oximeter sensor: A pulse oximeter is a non-contact device which can measure pulse and oxygen saturation (SpO₂) or regional oxygen saturation (rSO₂) in the blood. Typically, the sensor consists of two LEDs emitting light: one in the Red spectrum (RED-650 nm) and the other in Infrared (IR-950 nm). The SpO₂ levels are an estimated percentage of the amount of oxygenated Hemoglobin compared to the blood's total amount of Hemoglobin. The SpO₂ value is the oxygenated Hemoglobin level over the total Hemoglobin level as

$$Sp 02 = \frac{HbO 2}{Total Hb}$$

depending on the amount of oxygen in the blood, the ratio (R) between the absorbed Red light and IR light will be different. This ratio R is calculated as R=(AC_{RMSRED}/DC_{RED})/(AC_{RMSIR}/DC_{IR}). From this ratio, it is possible to calculate the oxygen level in blood Hemoglobin using an empirical or theoretical linear relationship. In-ear pulse oximeter is a viable technology solution for real-time detection of high-altitude hypoxia and acceleration induced hypoxia. The EWD prototype can be programmed to trigger signals. If rSO₂ goes below a particular baseline, it will trigger an analog signal for visual or audio and digital signal to integrate with a haptic feedback transducer. This EWD sensor can be programmed to calculate the time remaining before fatigue due to brain oxygen depletion sets in.

2. IMU: Miniature IMUs are based on MEMS accelerometers and gyroscopes; they are ubiquitously used from smartphones to aircraft. Given their low size, weight and power and cost (SWAP-C) and wide commercial availability, they provide an efficient way to measure human motion; IMUs can measure coupled head motion very accurately.

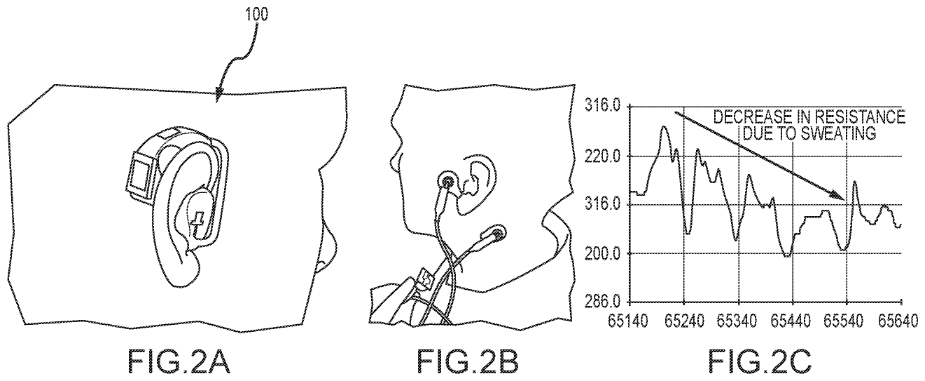

3. GSR sensor: Galvanic Skin Response (or electrodermal activity) refers to changes in the sweat gland activity correlated to the mental state or emotional arousal. The GSR sensor applies a constant voltage—usually 0.5 V—to the two electrodes that are in contact with the skin. The circuit also contains a minimal resistance compared to the skin resistance in series with the supply voltage and the electrodes. This voltage divider circuit (like an ohmmeter) is used to measure skin resistance (1/conductance). The raw GSR signal may contain various unwanted artifacts like high-frequency noise, rapid transition effects, temperature changes, etc. that should be removed via digital signal processing algorithms. A perspective view of EWD 100 with GSR electrodes (e.g., see GSR sensor 106 of FIG. 1A and FIG. 1B) worn by a user is shown in FIG. 2A. The GSR prototype built and tested by the inventors and the resulting sensor data is shown in FIG. 2B and FIG. 2C, respectively.

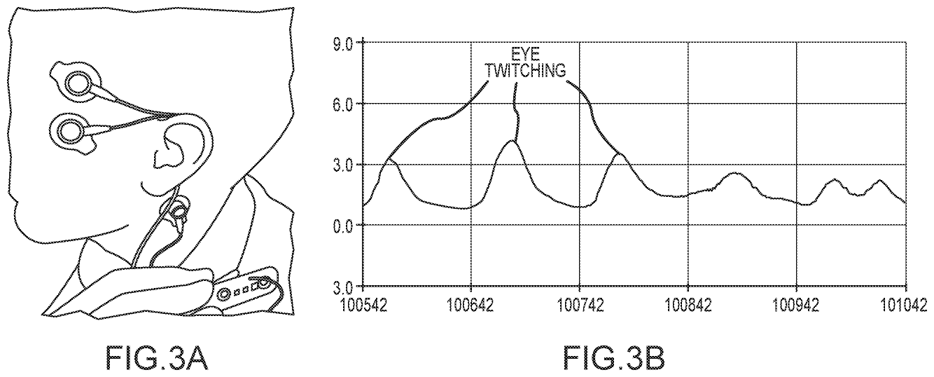

4. EMG Sensor: Electromyography (EMG) is an electrodiagnostic medicine technique for evaluating and recording the electrical activity produced by skeletal muscles. A low-cost filter and rectifier such as MyoWare AT-04-001 can be integrated into electronics that can capture raw EMG signal from muscles and convert it into rectified and integrated EMG signals. The EMG electrodes can be directly attached to the skin (e.g., via electrodes 140 of FIG. 1A and/or via electrodes 152 of FIG. 1D) or integrated into a fabric bodysuit or helmet. Researchers have shown a positive correlation between muscle tension in trapezius muscles and frontalis muscles to emotional stress, anxiety, tension headaches, and migraines. The prototype EMG sensor developed by the inventors and preliminary testing results for eye twitching are shown in FIG. 3A and FIG. 3B, respectively.

5. EKG/ECG Sensor: EKG or ECG records the heart's electrical activity using electrodes placed on the skin. Conventionally a 12-electrode system is used to measure heart electrical activity in 3 dimensions. Recently one lead (2 or 3 electrodes) EEG sensors have become available for personal use. Smartwatches like the apple watch now include ECG chips to record and display the EKG signal. The EWD may include a cardio-chip by Neurosky http://neurosky.com/biosensors/ecg-sensor/to acquire and process behind the ear EEG signal. It is noted that this single lead ECG sensor may not provide medical-grade ECG but help identify a) sinus rhythm-heart beating in a regular pattern, b) Atrial fibrillation-irregular beating of the heart between 50-120 BPM, c) Low (<50) or high (>120) heart rate.

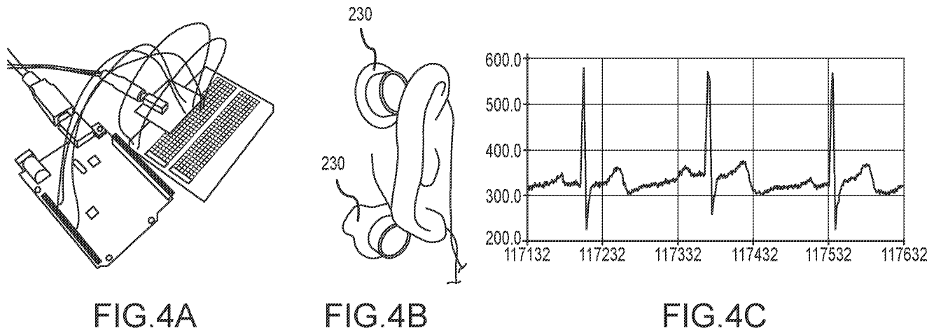

The inventors of the present disclosure designed and tested a behind the ear ECG system, as shown in FIGS. 4A through FIG. 4C. FIG. 4A shows the hardware/electronics for the ECG sensor. The ECG DSP chip was connected to electrodes (leads) 230 attached behind the ear (shown in FIG. 4B). The actual waveform (sinus rhythm) obtained from this system is shown in FIG. 4C.

6. EEG sensor: Electroencephalography (EEG) refers to the phenomenon of recording the electrical activity along the scalp, and Electroencephalogram (EEG) is referred to the recorded signals. It is the measure of voltage fluctuations/variations that occurred due to the flow of electrochemical currents in the brain's neurons. Typical medical-grade EEG systems use wet electrodes with 32 to 128 channel configurations. These EEG caps are bulky and cumbersome. Recently, Neurosky developed a low-cost, high-performance TGAT1 EEG signal processing chip that uses one lead configuration. This TGAT1 chip uses two dry electrodes to capture the most dominant EEG signals. These dry electrodes can be attached to the skull to acquire the EEG waveform.

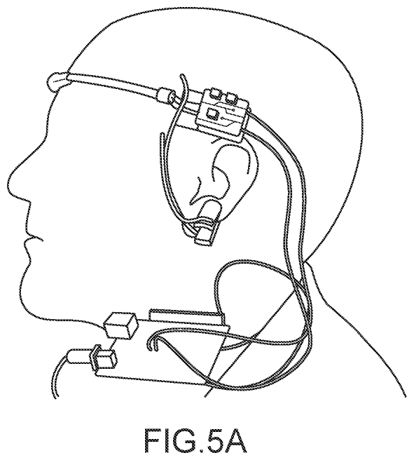

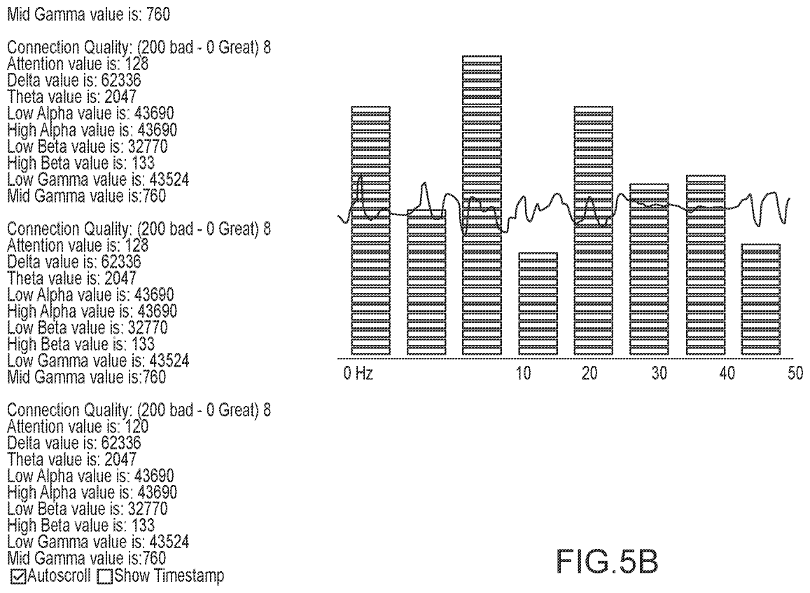

In embodiments, power spectrum analysis may be performed on alpha and theta waves to understand alertness, cognitive stress, and other neurological parameters. The EEG signal jumps can be used to detect eye blinks (that could be indicative of eye irritation, drowsiness, etc.). The inventors developed a single lead EEG system, as shown in FIG. 5A. The inventors could get the EEG signal and perform power spectral analysis, as shown in FIG. 5B. Electrodes 140 of FIG. 1A and/or electrodes 152 of FIG. 1D may be used as the EEG sensor.

It is noted that signal electrodes are needed for GSR, EMG, EKG, and EEG sensors. It is also possible to integrate the electrodes in a fabric, headband, scarf, or helmet worn by the operator.

Environmental sensing: The primary sensors in EWD 100 (Pulse Oximeter, Temperature, Heart rate, EMG, EKG, GSR, EEG, and IMU) can monitor various physiological signals. Micro-environment sensors like local pressure, radiation exposure, air quality, etc. can be integrated with the EWD 100.

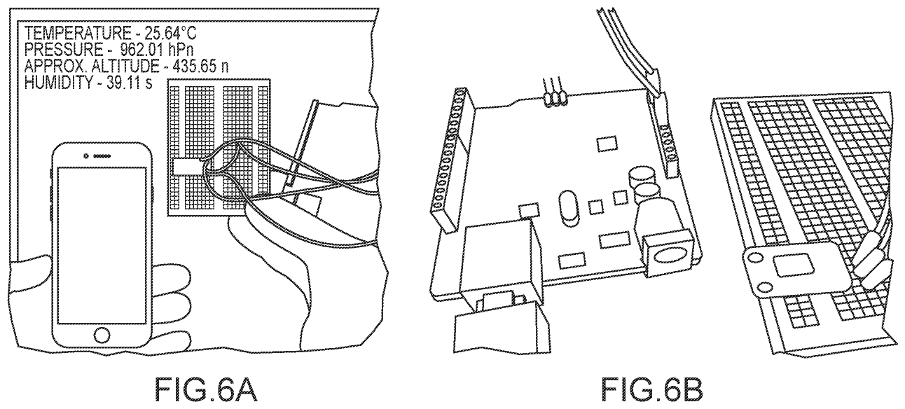

a) Barometer/Humidity sensor: This MEMS barosensor-BME280 can be integrated into EWD 100 to measure humidity, temperature, and cabin pressure/altitude. A simple benchtop prototype of this sensor built and tested by the inventors as part of the research is shown in FIG. 6A.

b) Gas Sensor/Air quality sensor: This sensor can be used for micro-environment air quality monitoring, in-cabin carbon monoxide and natural gas leakage detection, breath/alcohol checker, and early fire detection. The prototype setup for the gas sensor is shown in FIG. 6B.

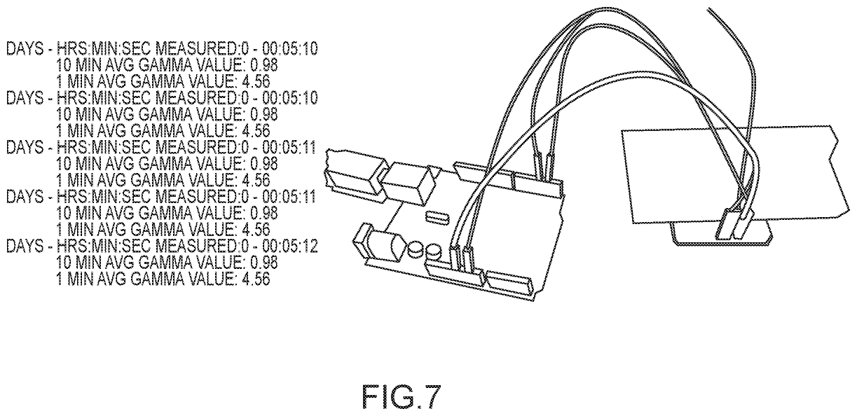

c) Radiation detection sensor: In embodiments, a radiation detection sensor may comprise a solid-state gamma radiation sensor such as that used by FTLAB (see http://allsmartlab.com/eng/294-2/). In embodiments, the radiation detection sensor is optimized for the low-level gamma detection up to 200 μSv/h. The radiation detection sensor can be characterized for radiation detection per datasheet specifications to add radiation sensing capability to EWD. The prototype and subsequent testing of radiation detection sensors (ADC values from the microcontroller) are shown in FIG. 7. It is noted that this sensor was tested with safe to handle radiation source Geiger counter card from United Nuclear. (http://unitednuclear.com/index.php?main_page=product_info&products_id=1005)

An EWD of the present disclosure may provide a modular, interoperable, and customizable earpiece with multiple chemical, radiation, and physiological sensors (IMU, Pulse Oximeter, EEG, Thermometer, GSR, etc.).

An EWD of the present disclosure may provide pre-symptomatic warning capability to monitor and enhance warfighter readiness, wellbeing, safety, and performance.

An EWD of the present disclosure may measure individual body temperature, breathing rate, heart rate, breathing and heart rate variability, blood/cerebral oxygen saturation, facial muscle movement, 3-axis and angular head acceleration/rotation rate/orientation.

An EWD of the present disclosure may wirelessly relay and or store (in remote or secluded areas) ABC signature information and alerts on CBRN exposure event, drowsiness, physical and thermal stress, exhaustion, fatigue, pulmonary distress, anxiety, consciousness, hypoxia, hyperventilation, head/neck loading and stress, acceleration atelectasis, toxic and infectious exposures/incapacitation etc. EWD 100 can be used to detect a CB adverse event as shown in Table 4 by capturing physiological parameters.

TABLE 4
Adverse chemical-biological (CB) events detected by EWD

| Physiological Parameter Measured | CB Exposure Symptom |
|---|---|
| Acceleration, angular velocity, elevation | Position, Cognitive distress, |
| data like total gain/loss steps | Physical distress, and |
| ascended/descended, flights | Emotional distress |
| ascended/descended, and elevation rate | |
| Cerebral oxygen saturation/Blood oxygen | Cardiac distress, Pulmonary |
| Heart Rate and intervals | distress, Cognitive distress, |
| Breathing Rate and intervals | Muscular Distress, and |
| Body temperature | Emotional distress |
| skin resistance, | Cognitive and emotional |
| measures skin conductance, changes in | distress |
| electrical (ionic) activity resulting from | |
| changes in sweat gland activity | |
| Electromyography Response (EMG | Muscular distress, |
| Sensor) | Cardiac and pulmonary |
| | distress |
| Presence of chemicals in environment | Environmental Toxicity |
| Presence of radiation in environment | Environmental Toxicity |

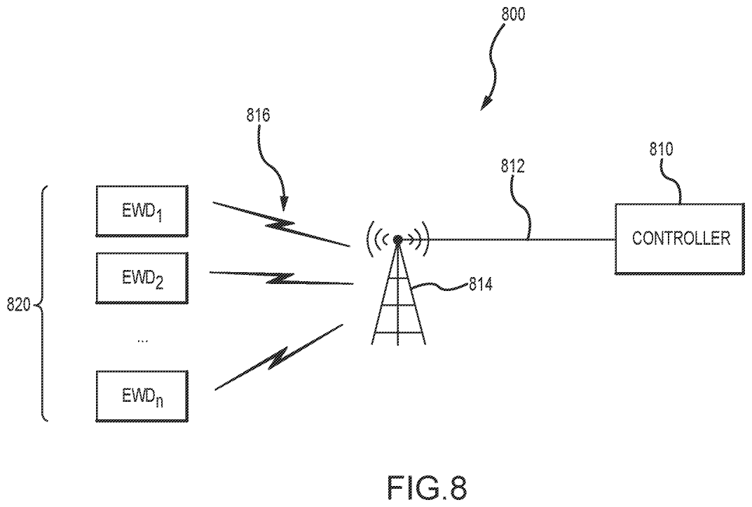

With reference to FIG. 8, a system 800 for monitoring personnel physiological parameters and environmental conditions using one or more EWDs 820 is illustrated, in accordance with various embodiments. In embodiments, system 800 comprises a controller node 810, an access node 814, and one or more EWDs 820. In embodiments, each EWD 820 may comprise a transmitter for transmitting data via data stream 816 to controller node 810 via access node 814. In embodiments, each EWD 820 transmits data to a local device (e.g., a smartphone or other device located with the user) which conveys sensor data to controller node 810 via access node 814. Access node 814 may communicate with controller node 810 via communication link 812.

In embodiments, access node 814 can be any network node configured to provide communication between EWDs 820 and controller node 810. Access node 814 may comprise a cell network, a satellite network, a radio base station, or any other type of network node suitable for transmitting data from EWDs 820 to controller node 810 and is not particularly limited.

Communication link 812 may use various communication media, such as air, space, metal, optical fiber, or some other signal propagation path including combinations thereof. Communication link 812 may be wired or wireless and use various communication protocols such as Internet, Internet protocol (IP), local-area network (LAN), optical networking, hybrid fiber coax (HFC), telephony, T1, or some other communication format—including combinations, improvements, or variations thereof. Wireless communication links can be a radio frequency, microwave, infrared, or other similar signal, and can use a suitable communication protocol, for example, Global System for Mobile telecommunications (GSM), Code Division Multiple Access (CDMA), Worldwide Interoperability for Microwave Access (WiMAX), Long Term Evolution (LTE), 5G NR, or combinations thereof. Communication link 812 may include Si communication links. Other wireless protocols can also be used. Communication link 812 can be a direct link or might include various equipment, intermediate components, systems, and networks. Communication link 812 may comprise many different signals sharing the same link.

Controller node 810 may be any network node configured to communicate information and/or control information over system 800. Controller node 810 may be a standalone computing device, computing system, or network component, and may be accessible using a communication interface connection (e.g., a wired or wireless connection), or through an indirect connection such as through a computer network or communication network. One of ordinary skill in the art would recognize that controller node 810 is not limited to any specific technology architecture, such as LTE or 5G NR, and can be used with any network architecture and/or protocol.

Physiological and environmental variables of an EWD user (e.g., a soldier, etc.) may be monitored via controller node 810 by those overseeing exercises. For example, controller 810 may receive sensor data from EWDs 820 where the sensor data is monitored for detecting dangerous, or potentially dangerous situations. For example, the EWD user may be withdrawn from the exercise, or moved to a safe location, if it appears that harm is likely.

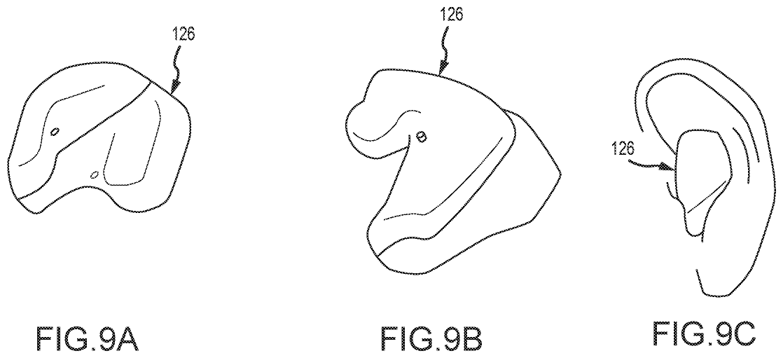

With reference to FIGS. 9A, 9B, and 9C, a prototype earpiece 126 for an EWD system is illustrated, in accordance with various embodiments.

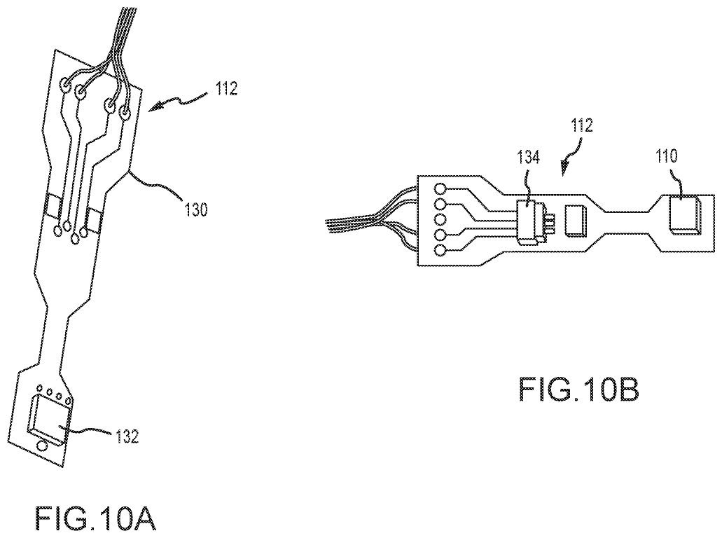

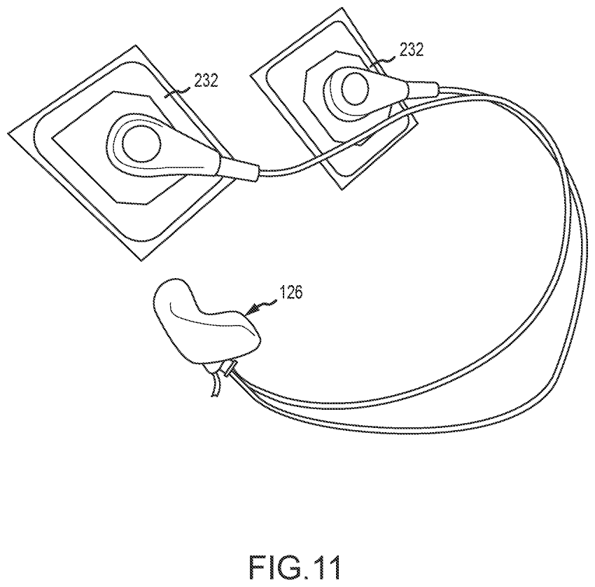

With reference to FIGS. 10A, and 10B a front-side view and a back-side view, respectively, of a printed circuit board (PCB) 130 are illustrated, in accordance with various embodiments. with PCB 130 may include an integrated pulse oximeter sensor 132 mounted to PCB 130. Moreover, PCB 130 may further include an integrated electrocardiogram sensor connector 134 mounted to PCB 130. Still further, PCB 130 may further include an integrated IMU sensor 110 mounted to PCB 130. In embodiments, PCB 130 is a flexible printed circuit board. PCB 130 can be integrated together with and installed on or in an earpiece 126 (see FIG. 1A). PCB 110 of FIG. 1A may comprise PCB 130, in accordance with various embodiments.

With reference to FIG. 11, EKG electrodes 232 are illustrated. The EKG electrodes 232 can be located for achieving a high signal to noise (S/N) ratio, for example by attaching EKG electrodes 232 to locations of a user without significant muscle mass under the skin, such as behind the ear or neck region.

While the principles of this disclosure have been shown in various embodiments, many modifications of structure, arrangements, proportions, the elements, materials and components, used in practice, which are particularly adapted for a specific environment and operating requirements may be used without departing from the principles and scope of this disclosure. These and other changes or modifications are intended to be included within the scope of the present disclosure.

The present disclosure has been described with reference to various embodiments. However, one of ordinary skill in the art appreciates that various modifications and changes can be made without departing from the scope of the present disclosure. Accordingly, the specification is to be regarded in an illustrative rather than a restrictive sense, and all such modifications are intended to be included within the scope of the present disclosure. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element.

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. Also, as used herein, the terms “coupled,” “coupling,” or any other variation thereof, are intended to cover a physical connection, an electrical connection, a magnetic connection, an optical connection, a communicative connection, a functional connection, and/or any other connection. When language similar to “at least one of A, B, or C” or “at least one of A, B, and C” is used in the specification or claims, the phrase is intended to mean any of the following: (1) at least one of A; (2) at least one of B; (3) at least one of C; (4) at least one of A and at least one of B; (5) at least one of B and at least one of C; (6) at least one of A and at least one of C; or (7) at least one of A, at least one of B, and at least one of C.

What is claimed is:

1. An in-ear wearable apparatus for monitoring physiological and environmental parameters of a person, comprising:
a housing;
a controller coupled to the housing;
a first plurality of physiological sensors coupled to the housing and configured to detect a plurality of physiological parameters; and
at least one environmental sensor coupled to the housing and configured to detect at least one environmental condition.

2. The in-ear wearable apparatus of claim 1, wherein the controller is configured to transmit the plurality of physiological parameters and the at least one environmental condition to a centralized computer.

3. The in-ear wearable apparatus of claim 1, wherein the first plurality of physiological sensors comprises at least two of the following: an IMU sensor, a pulse oximetry sensor, a GSR sensor, an EMG Sensor, an EKG sensor, and an EEG sensor.

4. The in-ear wearable apparatus of claim 3, wherein the at least one environmental sensor comprises at least one of the following: a barometer/humidity sensor, a gas sensor, and a radiation sensor.

5. The in-ear wearable apparatus of claim 4, wherein the first plurality of physiological sensors comprises the IMU sensor, the pulse oximetry sensor, the GSR sensor, the EMG Sensor, the EKG sensor, and the EEG sensor.

6. The in-ear wearable apparatus of claim 4, wherein the at least one environmental sensor comprises the barometer/humidity sensor, the gas sensor, and the radiation sensor.

7. The in-ear wearable apparatus of claim 1, further comprising an ear-mold and an in-ear sensor mounted to the ear-mold.

8. The in-ear wearable apparatus of claim 7, wherein the in-ear sensor comprises at least one of a pulse oximetry sensor, a temperature sensor, and a heart rate sensor.

9. The in-ear wearable apparatus of claim 7, wherein the housing is wearable behind an ear of the person ear and the ear-mold is wearable in the ear of the person.

10. The in-ear wearable apparatus of claim 1, further comprising a battery coupled to the housing, the battery configured to power the controller.

11. The in-ear wearable apparatus of claim 1, further comprising a flexible printed circuit board comprising an integrated pulse oximeter sensor.

12. The in-ear wearable apparatus of claim 11, wherein the flexible printed circuit board is mounted to an ear-mold of the in-ear wearable apparatus.

13. The in-ear wearable apparatus of claim 11, wherein the flexible printed circuit board further comprises an integrated electrocardiogram sensor connector.

14. The in-ear wearable apparatus of claim 11, wherein the flexible printed circuit board further comprises an integrated motion sensor.

15. A method of monitoring a physiological parameter and an environmental condition of a subject via an in-ear wearable apparatus, wherein the in-ear wearable apparatus includes a housing, a processor attached to the housing, a plurality of physiological sensors, and at least one environmental sensor, the method comprising:
obtaining physiological information from the subject via the plurality of physiological sensors, wherein the physiological information comprises at least one of the following: 3 axis head acceleration information, 3 axis rotation rate information, head orientation (roll, pitch, yaw) information, head angular acceleration information, cerebral oxygen saturation information, heart rate information, heart rate variability information, breathing rate information, breathing rate variability information, body temperature information, and vibration information; and
obtaining environmental information from the subject via the at least one environmental sensor, wherein the environmental information comprises at least one of the following: pressure information, attitude information, humidity information, temperature information, radiation information, and toxic gas information.

16. The method of claim 15, further comprising transmitting the physiological information and the environmental information to a device remotely located from the subject.

17. The method of claim 15, further comprising processing the physiological information and the environmental information to determine at least one of the following: a position of the subject, a cognitive distress of the subject, a physical distress of the subject, an emotional distress of the subject, a cardiac distress of the subject, a pulmonary distress of the subject, a muscular distress of the subject, and an environmental toxicity.
